(19) United States
(12) Patent Application Publication
(10) Pub. No.: US 2005/0182100 A1
(43) Pub. Date: Aug. 18, 2005

(54) Method of use

(76) Inventors: Ola Junghard, Molndal (SE); Ingela Wiklund, Molndal (SE)

Correspondence Address: WHITE & CASE LLP;PATENT DEPARTMENT, 1155 AVENUE OF THE AMERICAS, NEW YORK, NY, 10036, US

(21) Appl. No.: 10/508,755

(22) PCT Filed: Mar. 28, 2002
(86) PCT No.: PCT/SE02/00642

(51) Int. Cl. A61K 31/4439

(52) U.S. Cl. 514/338

(57) ABSTRACT

A method for treatment of sleeping disturbance to obtain improvement of sleeping, wherein a therapeutically effective dose of esomeprazole or an alkaline salt thereof is administered to a patient suffering therefrom. The method is applicable for instance in patients suffering from gastroesophagal diseases (GERD).

### FIELD OF INVENTION

The present invention relates to a method of treating sleeping disturbance in patients by administration of S-5-methoxy-2-[[[(4-methoxy-3,5-dimethyl-2-pyridinyl)methyl]sulfinyl]-1H-benzimidazole, known as esomeprazole, to obtain an improvement in sleeping and on a more general level symptom relief and health-related quality of life.

### BACKGROUND OF THE INVENTION

Gastric contents, frequently acidic, may give rise to dyspeptic symptoms. Most common symptoms are heartburn, acid regurgitation and chest pain. Other symptoms such as coughing, hoarsness, wheezing and asthma-like symptoms are also encountered. Heartburn is commonly reported. Up to 44% of Americans have heartburn at least monthly. Patients with these dyspeptic symptoms are said to suffer from gastro-esophageal reflux disease (GERD). It is estimated that about 25% of GERD sufferers seek medical advise and among these between 35% and 70% have none or minimal evidence of endoscopic esophagitis. More than one-third of the patents with heartburn report weekly symptoms and most patients have had their symptoms for more than one year. The primary impact of GERD is on a patient's day to day functioning and quality of life. Sleep disturbance is commonly encountered.

It is to be noted that dyspepsia is a common disorder and patients are seeing both gastroenterologists and general practicians because of it. Symtoms associated with dyspepsia are for instance upper abdominal pain/discomfort, heartburn, indigestion and sour stomach.

Therapeutic agents effective in the treatment of dyspepsia and GERD include gastric acid suppressing agents, such as H₂ receptor antagonists and proton pump inhibitors. Other agents of interest are antacids/alginates and prokinetic agents. These agents can be distinguished by their mechanisms of action, safety profile, pharmacokinetics and indications.

Antacid agents and alginates may be used alone in the treatment of heartburn. They have a short duration of action but are seen as inexpensive and safe. Antacid agents work locally through a neutralisation of gastric acid. Alginates further give some mechanical protection against reflux or gastric acid into the esophagasus. The main advantages of antacid agents and alginates are, that they provide fast relief of symtoms. The main disadvantage of antacid agents and alginates is that, dosing has to be repeated frequently to keep the patients free of symtoms, further that antacids in many cases do not provide symtom resolution, i.e. complete relief of symtoms.

H₂ receptor antagonists are widely prescribed for reducing gastric acid secretion systemically. Proton pump inhibitors, such as omeprazole, lansoprazole and pantoprazole are rapidly taking share from H₂ receptor antagonists. Omeprazole is known to offer significant gain over H₂ receptor antagonists in terms of symptom resolution, healing and prevention of relapse.

Proton pump inhibitors have in clinical studies been proven to be very effective in providing symtom resolution (usually within 24-48 hours) in patients with dyspepsia associated with gastric ulcers, duodenal ulcers, reflux esophagitis and gastroesophageal reflux without esophagitis. It is for instance established that omeprazole is superior to H₂ receptor antagonists regarding healing of gastroduodenal and esophageal lesions as well as providing dyspeptic symtom resolution in these conditions.

The S-enantiomer of omeprazole, having the generic name esomeprazole, is recently launched as a new generation of proton pump inhibitors. Esomeprazole shows further improvements in the treatment of GERD.

Omeprazole, i.e. the compound 5-methoxy-2-[[(4-methoxy-3,5-dimethyl-2-pyridinyl)methyl]sulfinyl]-1H-benzimidazole, and therapeutically acceptable salts thereof, are described in EP 5129. The specific alkaline salts of omeprazole are disclosed in EP 124 495.

Omeprazole is a sulfoxide and a chiral compound, wherein the sulfur atom being the stereogenic center. Thus, omeprazole is a racemic mixture of its two single enantiomers, the R- and S-enantiomer of omeprazole, herein referred to as R-omeprazole and S-omeprazole, the latter having the generic name esomeprazole. The absolute configurations of the enantiomers of omeprazole have been determined by an X-ray study of an N-alkylated derivative of the (+)-enantiomer in non-salt form. The (+)-enantiomer of the non-salt form and the (−)-enantiomer of the non-salt form were found to have R and S configuration, respectively, and the (+)-enantiomer of the magnesium salt and the (−)-enantiomer of the magnesium salt were also found to have R and S configuration, respectively. The conditions for the optical rotation measurement for each of these enantiomers are described in WO 94/27988.

Certain salts of single enantiomers of omeprazole and their preparation are disclosed in WO 94/27988. These compounds have improved pharmacokinetic and metabolic properties, which will give an improved therapeutic profile such as a lower degree of interindividual variation.

WO 96/02535 discloses a process for the preparation of the single enantiomers of omeprazole and salts thereof.

Proton pump inhibitors are susceptible to degradation/transformation in acid reacting and neutral media. In respect of the stability properties, it is obvious that the proton pump inhibitor must be protected from contact with acidic gastric juice by an enteric coating layer. There are different enteric coating layered preparations of proton pump inhibitors described in the prior art, see for example U.S. Pat. No. 4,786,505 (AB Hassle) and WO 96/01623 (Astra AB).

### SUMMARY OF THE INVENTION

It has been found according to the invention that administration of S-5-methoxy-2-[[(4-methoxy-3,5-dimethyl-2-pyridinyl)methyl]sulfinyl]-1H-benzimidazole, having the generic name esomeprazole, to patients affected by sleeping disturbance results in disappearance or great improvement of the symptoms. This applies especially to patients with GERD.

Esomeprazole is a pharmaceutical agent having the formula

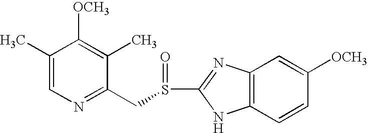

The active compound used according to the invention may be used in neutral form or in the form of an alkaline salt, such as for instance the Mg²⁺, Ca²⁺, Na²⁺ or K⁺ salts, preferably the Mg²⁺ salts.

The chemical name S-5-methoxy-2-[[(4-methoxy-3,5-dimethyl-2-pyridinyl)methyl]sulfinyl]-1H-benzimidazole and pharmaceutically acceptable salts thereof does not necessarily mean that the methoxy group of the benzimidazole moiety is in the 5-position but may as well be in the 6-position, or there may be mixtures of the two. This is due to equilibration in solution before the salts are formed in the solid state. The numbering is in accordance with the rules for nomenclature of organic chemistry, namely that the numbering of the atoms in the benzimidazole moiety should be done in such a way that the substituents should get the lowest possible number.

Esomeprazole can be administered orally, rectally or parenterally in neutral form or in the form of an alkaline or basic salt, such as for instance the Mg²⁺, Ca²⁺, Na⁺, or K⁺ salts, preferably the Mg²⁺ or Na⁺ salts.

The present invention is also applicable to other proton pump inhibitor compounds such as for instance omeprazole, lansoprazole, pantoprazole and rabeprazole.

While the effect on the symptoms of sleeping disturbance have been established in patients who have taken esomeprazole by the oral route, it is believed that the improved effect of esomeprazole is a systemic effect which is not dependent on what mode of administration that is used, and that accordingly the improved effect on sleeping and quality of life will be seen also with other routes of administration such as rectal or parenteral administration.

The commercially available pharmaceutical formulations of esomeprazole will normally be used also for treating sleeping disturbance and obtaining improved sleeping. Presently commercially available formulations marked under the tradename Nexium are based on esomeprazole magnesium salt in the form of enteric coating layered pellets filed in a capsule or multiple unit tablets comprising the same active ingredient.

Being a labile compound with poor storage stability at neutral or acid pH, esomeprazole formulations must be produced with great care. Examples of ways of producing stable formulations are given in e.g. EP 247 983 and EP 723 476.

The dose of esomeprazole to be administered in the treatment of sleeping disorders to obtain an improvement in sleeping will vary depending on factors such as the severity of the condition and the status of the patient. The dosage range at oral, rectal as well as i.v. administration may be in the interval from 1 to 100 mg per day. Normally, an amount of from 10 to 40 mg of esomeprazole daily and more preferred 20 mg and 40 mg is envisaged at oral administration.

The invention is further exemplified by the following case studies. During oral treatment with esomeprazole for acid related diseases such as non-erosive and erosive induced upper gastrointestinal disorders, evidence has accumulated that esomeprazole may be beneficial for treatment of sleeping disturbance especially in patients with GERD and endoscopy negative GERD. Some examples are presented below:

OBJECTIVES: Endoscopy-negative gastroesophageal reflux disease (GERD) lacks objective markers of disease severity. Evaluation of therapies for GERD must therefore rely on subjective measures, including patient self-report questionnaires, to measure the clinical effectiveness of therapeutic interventions. We aimed to evaluate the previously validated Gastrointestinal Symptoms Rating Scale (GSRS) and the Quality of Life in Reflux and Dyspepsia (QOLRAD) questionnaires for reliability and responsiveness to change over time.

METHODS: Patients (n=1143) with heartburn, but no esophagitis included in a randomized clinical trial assessing the effectiveness of active treatment with proton pump inhibitors over 4 wk were evaluated.

RESULTS: The test-retest reliability of both questionnaires over time was good to excellent (GSRS 0.53-0.69; QOLRAD 0.65-0.76), as was the responsiveness estimated by standardized response means (GSRS reflux dimension, −1.43; QOLRAD 0.81-1.43) and effect sizes (GRSR reflux dimension, −1.74; QOLRAD 0.82-1.56). The relationship between improvement in the GSRS reflux dimension score and the amount of clinical benefit as estimated by the patients themselves (based on the Overall Treatment Evaluation) suggested a minimally clinical relevant change is 0.5 on the seven graded scales applied. The importance rating indicated that an important change in the GSRS reflux dimension and the QOLRAD dimensions is equivalent to 1.0, and a very important change to 1.5.

CONCLUSIONS: The GSRS and QOLRAD are valid questionnaires that are reliable and sensitive to change. Both questionnaires should be suitable for use in clinical trials of therapeutic interventions for patients with heartburn.

(Am J Gastroenterol 2001;96:1998-2004. © 2001 by Am. Coll. of Gastroenterology)

Patients and Methods

A total of 1143 male and female patients, aged 18 to 80 yr, identifying their main symptom as a burning feeling rising from the stomach or the lower part of the chest up toward the neck (i.e., heartburn), were studied. All patients were included in a multi-center, double-blind, randomized, parallel group study. They completed quality of life questionnaires at baseline and during follow-up at 2 and 4 wk. All patients were treated with proton pump inhibitors for 4 wk (esomeprazole 20 mg daily, esomeprazole 40 mg daily, or esomeprazole 20 mg daily). The eligibility criteria stated that patients with endoscopically verified normal esophageal mucosa, a history of episodes of heartburn for 6 months or longer, and episodes of heartburn for 4 days or more during the last 7 days were eligible to enter the study. Excluded were patients with symptoms likely to be caused by the irritable bowel syndrome according to the Manning criteria, as were patients with alarm symptoms (unintentional weight loss, hematemesis, melena, jaundice, or any other sign indicating serious or malignant disease), or current or historical evidence of other gastrointestinal diseases and conditions (e.g., Zollinger-Ellison syndrome, esophageal stricture, more than five gastric erosions during the past 2 yr, duodenal or gastric ulcers within the past 2 yr, Barrett's metaplasia).

The two self-report questionnaires, GSRS and QOLRAD, were completed at baseline and at 2 and 4 wk of treatment. In addition, an Overall Treatment Evaluation (OTE) was completed at 2 and 4 wk. The baseline questionnaires were completed at the clinic before clinical examination. A standardized procedure for the administration of the questionnaires was adhered to throughout the study. Demographic and clinical characteristics were recorded in the Case Report Form as was severity and frequency of heartburn assessed by the clinician at baseline and during follow-up.

Gastrointestinal Symptom Rating Scale (GSRS)

The GSRS was specifically developed to address symptoms important to patients with general gastrointestinal complaints and has been extensively validated in previous studies (18, 20). The GSRS includes 15 items addressing different gastrointestinal symptoms and uses a 7-point Likert response scale with verbal descriptors. The response scale is designed to measure the amount of discomfort a patient has experienced (none at all, minor, mild, moderate, moderately severe, severe, and very severe). A higher score in a GSRS cluster indicates more discomfort. The recall period refers to the past week. The 15 items combine into five symptom clusters labeled: reflux, abdominal pain, indigestion, diarrhea, and constipation. The reflux dimension was identified as the most relevant dimension for the patient population in this study. From individual items within a cluster, a mean score is calculated. The GSRS has been applied in patients with heartburn with and without esophagitis (21-24).

Quality of Life in Reflux and Dyspepsia (QOLRAD)

The QOLRAD was specifically developed to monitor changes in health-related quality of life in patients suffering from heartburn and dyspepsia. The development, initial psychometric documentation and cross-sectional validity have previously been presented (19). The QOLRAD questionnaire has 25 items with five clinically relevant domains depicting emotional distress, sleep disturbance, food and drink problems, physical/social functioning, and vitality. The recall period refers to the past week. A 7-point Likert response scale is used to assess how much or how often the item described the feelings of the patient with respect to degree of distress (none at all, hardly any at all, a little, some, a moderate amount, a lot, a great deal) or frequency of the problem (none of the time, hardly any of the time, a little of the time, some of the time, quite a lot of the time, most of the time, all of the time). A higher score on the QOLRAD indicates less frequency in a domain. A mean score is calculated using items in each domain.

Overall Treatment Evaluation (OTE)

An anchor-based approach asking patients to provide a global rating of perceived symptom change over time, was used to estimate the minimal clinically relevant change in the GSRS reflux dimension (17, 25, 26). The OTE was included as an independent measure of change, in order to determine numerical changes in symptoms and health-related quality of life. It has previously been used to clarify the clinical relevance of changes in health-related quality of life in clinical trials (27), and to justify the validity of what is considered to be a minimally important change (28). The hierarchical scale first asks the patient, “Since treatment started, has there been any change in your symptoms of heartburn or acid regurgitation?” resulting in a response of better, about the same, or worse. If the patient responded “better” or “worse”, the patient was asked to rate the degree of positive or negative change using a 7-point Likert scale, indicating how much better or worse their conditions were (almost the same, hardly better/worse at all, changed a little, somewhat, moderately, a good deal, a great deal, or a very great deal). In addition to the GSRS reflux dimension, the global rating of symptom change was also related to changes in the QOLRAD dimensions in a similar fashion.

The OTE included a second question asking the patient to rate the importance of the change if they answered better or worse to the first question. The importance question which asked, “Is this change (better, worse) important to you in carrying out your daily activities?” utilized a 7-point Likert scale to measure the degree of importance (not, slightly, somewhat, moderately, very, and extremely important).

Methodological Issues

RELIABILITY. In order for a symptom or quality of life questionnaire to be reliable, the ratio of the variability in scores between patients to the total variability—the reliability coefficient—should be of acceptable magnitude, i.e., >0.60 (29-31). Non-responders were in this case defined as patients who had “no change” or “about the same, hardly better/worse at all” after the treatment period according to the OTE. Such an approach using non-responsive patients with heartburn to test the stability of a questionnaire has been applied previously (20).

RESPONSIVENESS OR SENSITIVITY TO CHANGE. One fundamental attribute of a measurement tool is its ability to detect changes in a patient's condition over time. In order to demonstrate that a measure is responsive to a treatment with known effectiveness, it must be administered to patients over a given time period. Provided the treatment is effective, patients should experience a change in their condition as measured by disease-specific questionnaires. There is no consensus regarding how best to assess the responsiveness to change. Hence, various approaches have been reported (24). The traditional ways of measuring responsiveness are to calculate 1) the effect size by dividing the mean change by the standard deviation at baseline (32 and 2) the standardized response mean, which is the mean change divided by the standard deviation of the change. This latter method preserves the relation to a statistical test, whereas the former anchors the changes against the variability in the sample.

Statistical Analysis

To evaluate reliability, the within patient and between patient mean square were calculated by two-way analysis of variance (ANOVA) with the factors of patients and week (baseline and 2 and 4 wk). The calculation used data from those patients who reported no change during the 4 wk of treatment according to the OTE. The reliability coefficient is estimated as:

$$R = \frac{BMS - WMS}{[ BMS + ( {K}_{0} - 1 ) WMS ]}$$

where BMS and WMS are the mean square values between patients and within patients, respectively, and K₀ denotes the number of repeated time measurements (33). The procedure PROC GLM within SAS was used for the calculations. Values off R<0.4 may be taken to represent poor reliability, values >0.75 excellent reliability, and values in between, fair to good reliability (30, 33).

The clinical relevance was explored as change score correlation, using Pearson's correlation coefficient. This was done by correlating the change from baseline to 4 wk for the clinician rating of severity and frequency of heartburn with the change in the QOLRAD domains.

The perceived change according to the OTE was classified as none, small, moderate, and large (26). This system classifies patients who were about the same or “almost the same, hardly better/worse at all” as unchanged, those patients indicating a little or somewhat better/worse are classified as having a small OTE change, those indicating moderately or a good deal better/worse, as having a moderate OTE change, and those indicating a great deal or very great deal better/worse, as having a large OTE change (28). This collapsed OTE scale was given scores from −3 (for large negative change) to 3 (for large positive change) with zero for unchanged patients. Linear regression was used to determine the change in each dimension corresponding to a one unit change in the collapsed OTE scale. The change for the GSRS reflux cluster, which is directly related to the symptoms under treatment, is taken to represent a minimally relevant change.

The patient importance rating of change was determined by comparing the GSRS clusters and QOLRAD domains to the OTE importance scale. We collapsed the importance rating into 4 classifications: not important (not important), slight/moderately important (slightly, somewhat, or moderately important), important (important), and very important (very or extremely important).

Results

Patients characteristics and clinical data are summarized in Table 1. The severity of heartburn was on average moderate, with over 50% of the patients having daily symptoms, and almost half having a duration of symptoms >5 yr.

Reliability

The evaluation of reliability utilized the 73 patients who reported no change at both 2 and 4 wk of treatment. Table 2 shows the estimated reliability (intraclass correlation coefficients) for the GSRS clusters and the QOLRAD domains. Reliability ranged from 0.53 to 0.69 for the GSRS and 0.65 to 0.76 for the QOLRAD. Using predefined criteria, the reliability was good to excellent for most scales.

Change Score Correlations

The correlations between the change in the clinicians rating of severity and frequency of heartburn and the change in the QOLRAD domains are presented in Table 3. All correlations indicated a moderate relationship between relief of heartburn and improved quality of life. At 4 wk, the range was from 0.27 to 0.50 (p<0.0001). The correlations at 2 wk were very similar (data not shown).

TABLE 1
Demographic and Clinical Characteristics at Baseline

| | |
|---|---|
| Age (yr), mean (SD) | 48.3 (14.0) |
| Age (yr) | |
| 16-50 | 57* |
| 51-60 | 20 |
| 61-80 | 23 |
| Gender, males | 44 |
| Race, caucasian | 98 |
| Employed/self-employed, yes | 59 |
| Clinician rated frequency | |
| of heartburn | |
| 4 days/wk | 24 |
| 5-6 days/wk | 20 |
| 7 days/wk | 56 |
| Clinician rated severity of | |
| heartburn | |
| Mild | 22 |
| Moderate | 59 |
| Severe | 18 |
| History of heartburn | |
| <12 mo | 12 |
| 1-5 yr | 39 |
| >5 yr | 48 |

Patients with both a baseline and a subsequent visit (a - I 143).
*All values in this column are percentages.

Responsiveness

Table 4 presents the standardized response mean and the effect size at 4 wk for the GSRS and QOLRAD. The standardized response mean showed that, in addition to the GSRS reflux dimension, sizeable changes were also detected in the abdominal pain and indigestion dimensions. As expected, the responsiveness of the GSRS to constipation and diarrhea was lower. The responsiveness of the QOLRAD was excellent in all dimensions, in particular with regard to food and drink problems. Effect sizes were almost identical to the standardized response means for both questionnaires (Table 4). The results were very similar at 2 wk (data not shown).

The effect size was large for GSRS reflux and for all of the QOLRAD domains according to the definition of Cohen, which states that an effect size ≧0.5 indicates moderate sensitivity and an effect size of 0.8 indicates a large responsiveness to change (32).

Minimally Relevant Change

The changes in GSRS symptom clusters and QOLRAD domains in relation to the magnitude of the perceived improvement as defined by the OTE are presented in Table 5.

TABLE 2
Test-Retest Reliability Intraclass Correlation Coefficient (ICC)
in Patients Reporting No Change According to the Overall Treatment
Evaluation (OTE)

| | ICC | | ICC |
|---|---|---|---|
| GSRS | (n = 73) | QOLRAD | (n = 73) |
| Diarrhea | 0.69 | Emotional distress | 0.75 |
| Indigestion | 0.65 | Sleep disturbances | 0.76 |
| Constipation | 0.60 | Food/drink problems | 0.76 |
| Abdominal pain | 0.53 | Physical/social functioning | 0.76 |
| Reflux | 0.59 | Vitality | 0.65 |

TABLE 3
Change Score Corre1ations: Change in Clinician Rated Severity
and Frequency of Heartburn, and Change in QOLRAD Total and
Dimension Scores*

| | Severity | Frequency |
|---|---|---|
| | (n = 1108) | (n = 1108) |
| QOLRAD | r Value | r Value |
| Emotional distress | 0.43 | 0.34 |
| Sleep disturbances | 0.41 | 0.30 |
| Food/drink problems | 0.50 | 0.47 |
| Physical/social functioning | 0.39 | 0.27 |
| Vitality | 0.44 | 0.39 |

All correlations p < 0.0001.
r value Pearson correlation coefficient.
*Baseline to 4 wk.

The difference between adjacent OTE classes (small no change, moderate-small change, or large-moderate change) was approximately 0.5 score units for the GSRS reflux dimension (scale from 1-7). This suggests a minimally relevant change of 0.5. The QOLRAD exhibited similar results for all dimensions with the exception of the physical/social domain. The results were very similar at 2 wk (data not shown).

Table 6 shows the estimated change in GSRS reflux dimension and QOLRAD domains compared to a one-unit improvement in the collapsed OTE classification. All changes were statistically significant at the p<0.05 level. The change for the GSRS reflux dimension was 0.51 at 4 wk. This supports 0.5 as an approximate value for a minimally relevant is change. The results were also very similar at 2 wk (data not shown).

Patient Importance Rating of Change

Table 5 shows the change in GSRS and QOLRAD scores from baseline to 4 wk by the patient's rating of the importance of the change for carrying out daily activities as defined by the OTE. For the GSRS reflux dimension and for the QOLRAD dimensions, the change scores showed a consistent trend with increasing importance rating. An important change as defined by the patients equals approximately I score unit change in the QOLRAD dimensions, and in the GSRS reflux dimension. The results were similar at 2 wk (data not shown).

TABLE 4
Standardized Response Mean (SRM) and Effect Sizes at 4 Weeks

| | | Effect | | | Effect |
|---|---|---|---|---|---|
| GSRS | SRM (SD of change) | Size | QOLRAD | SRM (SD of change) | Size |
| Diarrhea | −0.15 (1.15) | −0.16 | Emotional distress | 1.24 (1.48) | 1.25 |
| Indigestion | −0.86 (1.24) | −0.83 | Sleep disturbances | 1.13 (1.46) | 1.12 |
| Constipation | −0.37 (1.10) | −0.34 | Food/drink problems | 1.43 (1.43) | 1.56 |
| Abdominal pain | −0.87 (1.19) | −0.93 | Physical/social func. | 0.81 (1.24) | 0.82 |
| Reflux | −1.43 (1.45) | −1.74 | Vitality | 1.24 (1.51) | 1.45 |

Discussion

Evaluations of health-related quality of life facilitate a translation of clinical benefits into outcomes of significance to patients. In patients with symptomatic heartburn without esophagitis, the evaluation of new therapies relies entirely upon subjective patient-based outcomes. There is no independent way of deriving confirmation of the degree of distress and dysfunction induced by symptoms. Standardized methods that address symptoms and health-related quality of life must therefore be used to monitor the patient's response to treatment. The development of subjective selfreport questionnaires for symptoms and quality of life assessment requires rigorous psychometric evaluation if these instruments are to be confidently used to assess treatment efficacy endpoints in clinical trials of new therapeutics (34-36). Traditionally, psychometric documentation has been limited to cross-sectional issues of item selection and domain construction, with the resulting domain intercorrelations, internal consistency, reliability, convergent validity, and discriminant validity. Although cross-sectional validity is a necessary precondition to the use of a questionnaire, it does not guarantee that the instrument will perform well when used in an actual clinical trial setting. Neither does it guarantee that the instrument will be able to detect changes that patients rate as significant. Thus, less attention has been paid to the psychometric documentation of test-retest reliability and responsiveness to change despite the fact that the latter feature in particular is crucial to an instrument's utility in clinical trials (37, 38).

This study was not ideal for assessing reliability owing to the relatively small proportions of patients who did not respond to treatment. Strictly, the estimated reliability applies only to this limited group of non-responding patients. However, the baseline sample variances for the non-responders were similar to the corresponding baseline sample variances for the whole study population. Thus, assuming the within patient variances for non-responders are representative for the study population, the reliability estimates should be applicable to the patient group under study. The reliability coefficients did suggest that the GSRS and QOLRAD are reliable instruments, as has been previously documented using similar technique (20).

Two different measures—the standardized response mean and the effect size used to assess responsiveness—yielded similar results, documenting the responsiveness of both questionnaires. The magnitude of the estimated responsiveness reflects the know efficacy of the treatment (i.e., in this case, treatment of symptomatic heartburn with proton pump inhibition). In another study, the effect size at 4 wk of treatment with proton pump inhibitors in the GSRS reflux dimension was 1.2 compared to 0.5 observed in the placebo aim, indicating a large effect (21). As expected, the responsiveness was lower for symptom clusters not associated with heartburn (i.e., symptoms of the lower gastrointestinaltract).

TABLE 5
Changes in GSRS and QOLRAD Dimension Scores From Baseline to 4 Weeks by Patient Rating of the Magnitude of
the Change As Well As the Importance of the Change.

| | | | | | Importance Rating | | | |
|---|---|---|---|---|---|---|---|---|
| | | | | | | Slight/ | | |
| | Change Rating | | | | Not | Moderately | | Very |
| | None | Small | Moderate | Large | Important | Important | Important | Important |
| GSRS | | | | | | | | |
| Dimension | n = 133 | n = 69 | n = 171 | n = 702 | n = 171 | n = 150 | n = 183 | n = 567 |
| Diarrhea | −0.01 (1.23) | 0.04 (1.18) | −0.32 (1.04) | −0.23 (1.11) | −0.09 (1.19) | 0.17 (1.07) | −0.17 (1.01) | −0.26 (1.16) |
| Indigestion | −0.60 (1.12) | −0.78 (0.97) | −1.04 (1.16) | −1.23 (1.24) | 0.65 (1.13) | −0.90 (1.22) | −0.95 (1.13) | −1.31 (1.23) |
| Constipation | −0.24 (1.32) | −0.38 (1.01) | −0.37 (0.99) | −0.48 (1.08) | −0.31 (1.25) | −0.38 (1.01) | −0.42 (1.07) | −0.47 (1.07) |
| Abdominal/ | −0.66 (1.20) | −0.65 (1.01) | −0.82 (1.02) | −1.27 (1.14) | −0.74 (1.16) | −0.87 (1.03) | −0.99 (1.11) | −1.27 (1.16) |
| Pain | | | | | | | | |
| Reflux | −0.99 (1.31) | −1.59 (1.22) | −1.68 (1.24) | −2.51 (1.29) | −1.20 (1.33) | −1.88 (1.19) | −2.13 (1.35) | 2.47 (1.34) |
| QOLRAD | | | | | | | | |
| Dimension | n = 140 | n = 71 | n = 175 | n = 721 | n = 178 | n = 159 | n = 191 | n = 580 |
| Emotional | 0.83 (1.18) | 1.51 (1.32) | 1.78 (1.23) | 2.18 (1.43) | 0.93 (1.17) | 1.47 (1.20) | 1.72 (1.18) | 2.37 (1.44) |
| Sleep | 0.75 (1.17) | 1.41 (1.31) | 1.63 (1.23) | 1.94 (1.43) | 0.88 (1.24) | 1.37 (1.23) | 1.57 (1.17) | 2.09 (1.45) |
| Food/drink | 0.85 (1.11) | 1.35 (1.20) | 1.78 (1.10) | 2.50 (1.32) | 1.02 (1.17) | 1.83 (1.10) | 1.93 (1.16) | 2.57 (1.37) |
| Phys/soc | 0.35 (0.93) | 0.79 (1.27) | 1.00 (1.02) | 1.24 (1.22) | 0.41 (0.92) | 0.73 (0.93) | 0.92 (1.03) | 1.40 (1.26) |
| Vitality | 0.77 (1.31) | 1.34 (1.30) | 1.58 (1.29) | 2.32 (1.38) | 0.97 (1.34) | 1.69 (1.20) | 1.74 (1.43) | 2.38 (1.39) |

Change rating and importance rating expressed as change in the mean (SD).

TABLE 6
Estimated Change in GSRS Reflux Dimension and QOLRAD Scores
Due to One-Unit Improvement in Collapsed Overall Treatment
Evaluation*.
GSRS

| Dimension | Estimate (STD error) |
|---|---|
| Reflux | −0.51 (0.03) |

QOLRAP

| Dimensions | Estimate (STD error) |
|---|---|
| Emotional distress | 0.45 (0.03) |
| Sleep disturbances | 0.41 (0.03) |
| Food/drink problems | 0.54 {0.03) |
| Physical/social functioning | 0.33 (0.03) |
| Vitality | 0.51 {0.03) |

*No change, small moderate, and large improvement at 4 wk.

The determination of the minimally clinical relevant change in clinical trials is a critical issue (17, 30, 38, 39). We based our analysis on the method employed by Juniper (26) and found that a minimal clinically relevant change was 0.5. This is consistent with findings for other instruments utilizing 7-point Likert scale, where the minimally important change has been estimated to be 0.5 unit (26, 28, 40), while a change score of approximately 1-5 units represented a large change in quality or life (26). One possible criticism of the approach taken in the present study was the selection of cut-off points used to classify the OTE effect. However, others have used similar modified approaches (17, 41).

Large change scores were found for the GSRS reflux dimension, and all of the QOLRAD domains. The observed changes were smaller in the physical/social dimension of QOLRAD. This is not surprising, because half of the patients had suffered from heartburn for 5 yr or more. With a long duration of disease, patients may adapt to their situation by avoiding activities that provoke symptoms (42). Compared with the effect sizes for antihypertensive therapy, which range from 0.01 to 0.3 (43), the present findings show, that large changes in the direction of better can be detected.

Beyond the evaluation of clinical relevant change is the question of the importance of a change to the patient. The patient must be the person that determines what is important to them, because the agreement between clinician and patient ratings of health-related quality or life is usually poor (44). This is one of the first studies that has tried to link what patients perceive as an important change in symptoms to changes in quality of life, an issue that is particularly pertinent in GERD where the evaluation of treatment effect relies on patient reporting. Thus, the effects of therapy on symptom relief and health-related quality of life are important considerations in the treatment of patients with symptomatic heartburn and should be considered as primary end-points in clinical trials in this patient population.

The association between relief of symptoms and health-related quality of life was studied by correlating the change in reflux symptoms rated by the clinician with the change in health-related quality of life as measured by the patient. There was a clear relationship between symptom resolution and improvement in QOLRAD domains, which supports the clinical validity of the health-related quality of life instrument. Food and drink problems, which may be related to acid secretion, were most strongly correlated with symptom relief. These findings suggest that the symptomatic benefits of treatment are directly reflected by an enhanced health-related quality of life. Both the GSRS reflux dimension and the QOLRAD have good reliability and are responsive to change in symptomatic heartburn. For these questionnaires, it is possible to quantify a minimally relevant change as well as the importance of change scores.

1. A method for treatment of sleeping disturbance, wherein the method comprises administering a therapeutically effective amount of S-5-methoxy-2-[[(4-methoxy-3,5-dimethyl-2-pyridinyl)methyl]sulfinyl]-1H-benzimidazole (esomeprazole) or a pharmaceutically acceptable salt thereof to a patient in need thereof.

2. A method for improving sleep in a patient suffering from gastroesophagal reflux disease, wherein the method comprises administering a therapeutically effective amount of S-5-methoxy-2-[[(4-methoxy-3,5-dimethyl-2-pyridinyl)methyl]sulfinyl]-1H-benzimidazole (esomeprazole) or a pharmaceutically acceptable salt thereof a patient in need thereof.

3. The method according to any one of claims 1 or 2, wherein the esomeprazole is administered in the form of an alkaline salt selected from the group consisting of Mg²⁺, Ca²⁺, Na²⁺ and K²⁺ salts.

4. The method according to any one of claims 1 or 2, wherein the salt is a magnesium salt.

5. The method according to one any of claims 1 or 2, wherein the salt is a magnesium salt.

6. The method according to any one of claims 1 or 2, wherein the esomeprazole or an alkaline salt thereof is administered orally.

7. The method according to any one of claims 1 or 2, wherein the esomeprazole or an alkaline salt thereof is administered parenterally.

8. The method according to any one of claims 1 or 2, wherein the esomeprazole or an alkaline salt thereof is administered in a dose of from 1 to 100 mg daily.

9. The method according to claim 8, wherein the esomeprazole or the alkaline salt thereof is administered in a dose of from 10 to 40 mg daily.

10. (canceled)

11. (canceled)
